(19) United States
(12) Patent Application Publication
(10) Pub. No.: US 2015/0132818 A1
(43) Pub. Date: May 14, 2015

(54) GLYCOSYLATION METHOD OF ALGAE OR AGRICULTURAL BY-PRODUCTS COMPRISING HIGH-PRESSURE EXTRUSION PULVERIZATION STEP

(71) Applicants: Do-Hyung Kang, Ansan-si (KR); Hyeon-Yong Lee, Chuncheon-si (KR); Woon-Yong Choi, Chuncheon-si (KR); Choon-Geun Lee, Chuncheon-si (KR); Yong-Chang Seo, Chuncheon-si (KR); Ji-Seon Kim, Seoul (KR); Chi-Ho Song, Chuncheon-si (KR); Kyung-Hwan Jung, Seoul (KR); Sang-Eun Lee, Cheongju-si (KR)

(72) Inventors: Do-Hyung Kang, Ansan-si (KR); Hyeon-Yong Lee, Chuncheon-si (KR); Woon-Yong Choi, Chuncheon-si (KR); Choon-Geun Lee, Chuncheon-si (KR); Yong-Chang Seo, Chuncheon-si (KR); Ji-Seon Kim, Seoul (KR); Chi-Ho Song, Chuncheon-si (KR); Kyung-Hwan Jung, Seoul (KR); Sang-Eun Lee, Cheongju-si (KR)

(73) Assignee: DAE-A Intellectual Property Consulting

(21) Appl. No.: 14/395,440

(22) PCT Filed: May 2, 2012
(86) PCT No.: PCT/KR2012/003424

§ 371 Date: Jan. 28, 2015

(30) Foreign Application Priority Data
Apr. 17, 2012 (KR) 10-2012-0039981

(51) Int. Cl. C13K 1/00; C12P 19/14; C12P 7/10; C12P 19/02

(52) U.S. Cl. CPC C13K 1/00; C12P 19/02; C12P 19/14; C12P 7/10; C12P 2201/00
USPC 435/165; 435/99; 435/95; 127/36

(57) ABSTRACT

Disclosed is a method of saccharifying biomass, such as algae or agricultural by-products by performing a high-pressure extrusion pulverization process for the biomass, such as algae or agricultural by-products, and more particularly to a method of saccharifying biomass, which includes homogenizing and crushing algae or agricultural by-products, and extruding the algae or agricultural by-products through a micro-diameter tube to pulverize the algae or agricultural by-products. Non-biodegradable polymers, such as cellulose, which is a polysaccharide included in biomass, such as algae or agricultural by-products, hemicelluloses, starch, and complex polysaccharide, are hydrolyzed at high glycosylation efficiency through an eco-friendly pretreatment process using water.

### TECHNICAL FIELD

The present invention relates to a method of saccharifying biomass, such as algae or agricultural by-products by performing a high-pressure extrusion pulverization process for the biomass, such as algae or agricultural by-products. More particularly, the present invention relates to a method of saccharifying biomass, which includes homogenizing and crushing algae or agricultural by-products, and extruding the algae or agricultural by-products through a micro-diameter tube to pulverize the algae or agricultural by-products.

### BACKGROUND ART

As human beings are rapidly increased and developed, foods have been depleted, and the energy shortage resulting from the indiscriminate use of coal fuel, and high oil price are caused. Accordingly, a big challenge of the development of substitute energy is given to human beings.

To accept the challenge, bio energy industries have been developed. Among them, sugar, which is the important source material in a bioethanol field, can be extensively used in algae or agricultural by-products. In addition, the sugar used in a food industry has been produced at a high cost. Particularly, to produce the sugar from sugar cane and sugar beet serving as main source materials of the sugar, chemical treatments must be accompanied. Accordingly, the chemical treatments cause environment pollution. In order to overcome the environment pollution, significant manpower and economical loss are inevitable.

Recently, in food industry fields and bio energy production fields various researches and studies have been performed on the development of a pretreatment process, an enzyme process, and new enzymes and micro-organisms for alcohol fermentation. However, the progress of researches and studies through an eco-friendly and efficient pretreatment process is in a low level, thereby causing many economical problems.

In addition, according to the related art, to prepare sugar, sugar cane and sugar beet are centrifuged (powdered), refined, and crystallized. In this case, a chemical process, which adds lime to remove impurities, may be interposed between centrifuging and refining processes. However, the above method causes a great economical problem, because of using food resources.

In the production of bio energy, a chemical pretreatment process is performed by using acid/alkali, and the researches and studies on the chemical pretreatment process have been most actively performed. For example, a method of producing bio energy using agricultural by-products, which is in a commercialization step, includes the steps of adding acid, such as sulfuric acid, to a source material, decomposing cellulose at a high temperature and high pressure, performing a neutralizing process by alkali, and performing enzyme treatment to decompose remaining cellulose, so that sugar can be obtained, and producing bio energy by fermenting the obtained sugar. In particular, Korean Unexamined Patent Publication No. 2010-0093253 discloses a method of pretreating and saccharifying marine algae biomass.

The pretreatment in the production of the bio energy must dependent on acid/alkali treatment and high-energy physical pretreatment due to the characteristics of a source material. In addition, the yield rate of saccharified materials according to the pretreatment represents a lower value as compared with investment cost. Further, the chemical pretreatment process has the greatest disadvantage in that a neutralization process of neutralizing an acid treatment result must be performed as a subsequent process of the pretreatment process. Further, in the case of the pretreatment process by acid, the high temperature and pressure conditions create furuals and furans serving as toxic properties to enzymes during the pretreatment process, thereby degrading the production efficiency of bio energy.

Accordingly, studies and researches have performed with respect to the pretreatment process using pure water rather than a pretreatment method by acid to hydrolyze sugar. Differently from the chemical pretreatment process, in the pretreatment process using water, the removal of acid through a neutralization process is required, and an inhibitor of enzyme is not produced, which represents an eco-friendly effect. Accordingly, the pretreatment process using water is applicable to whole industries including a food industry. However, since the pretreatment process only using water represents a low pretreatment speed and requires high energy to be introduced, the production cost can be increased. In particular, the conversion yield rate of glucose to be fermented is represented as a low value, so that a great amount of ethanol cannot be obtained in the final stage.

Accordingly, there are continuously required researches and studies on a method of saccharifying algae or agricultural by-products capable of representing superior glycosylation efficiency while solving a problem related to conventional chemical acid/alkali using water through the pretreatment process using water.

### DISCLOSURE

### Technical Problem

Therefore, inventors of the present invention have continuously tried to perform researches and studies on the development of a pretreatment method capable of producing eco-friendly and efficient monosaccharides, which are required in bio energy and food industries, from biomass, such as algae or agricultural by-products, by using pure water. As a result, the inventors complete the present invention by discovering that saccharified materials can be obtained at a high yield rate if the algae or agricultural by-products are homogenized and crushed, and extruded through a pipe having a micro-diameter by applying press to the pipe.

An object of the present invention is to provide a method of continuously preparing sugar at a high yield rate by pulverizing through an extrusion process without acid/alkali treatment.

### Technical Solution

In order to accomplish the above object, there is provided a method of saccharifying algae or agricultural by-products. The method includes 1) homogenizing and crushing the algae or agricultural by-products, and 2) extruding the algae or agricultural by-products that are crushed.

According to the present invention, a saccharified material may be obtained by performing enzyme-treatment for the extruded algae or agricultural by-products.

Further, there is provided a method of preparing bioethanol, which includes fermenting a saccharified material obtained through the method.

### Advantageous Effects

As described above, according to the present invention, non-biodegradable polymers, such as cellulose, which is a polysaccharide included in biomass, such as algae or agricultural by-products, hemicelluloses, starch, and complex polysaccharide can be hydrolyzed at high glycosylation efficiency through an eco-friendly pretreatment process using water.

In particular, according to the present invention, since only water is used, the neutralization process can be omitted. Further, since the features of the continuous pretreatment process can be represented, processes can be simplified, so that the low-cost and high efficiency can be expected.

In addition, since the saccharified materials produced according to the method of the present invention do not contain materials, such as furuals and furans, to degrade fermentation, the saccharified materials produced according to the method of the present invention can be widely applied to a food industry as well as a bio energy industry.

### DESCRIPTION OF DRAWINGS

FIG. 1 is a flowchart showing a method of saccharifying algae or agricultural by-products according to the present invention.

FIG. 2 is a graph showing the conversion efficiency of glucose resulting enzyme-saccharification of ulva pertusa kjellman according to a second experimental example.

FIG. 3 is a graph showing the conversion efficiency of glucose resulting enzyme-saccharification of gulfweed according to the second experimental example.

FIG. 4 is a graph showing the conversion efficiency of glucose resulting enzyme-saccharification of a barley stem according to the second experimental example.

FIG. 5 is a graph showing the conversion efficiency of glucose resulting enzyme-saccharification of a rape stem according to the second experimental example.

FIG. 6 is a graph showing ethanol production according to the fermentation of ulva pertusa kjellman in a third experimental example.

FIG. 7 is a graph showing ethanol production according to the fermentation of gulfweed in the third experimental example.

FIG. 8 is a graph showing ethanol production according to the fermentation of a barley stem in the third experimental example.

FIG. 9 is a graph showing ethanol production according to the fermentation of a rape stem in the third experimental example.

FIG. 10a is a graph showing a result obtained by analyzing ulva pertusa kjellman extruded in a first embodiment using a DLS nano-particle analyzer.

FIG. 10b is a graph showing a result obtained by analyzing a rape stem extruded in a first embodiment using the DLS nano-particle analyzer.

FIG. 11a is an SEM photograph showing a tissue surface of an ulva pertusa kjellman sample crushed using a homogenizer in the first embodiment.

FIG. 11b is an SEM photograph showing a tissue surface of an ulva pertusa kjellman sample crushed using a homogenizer in the first embodiment and subject to an extrusion process.

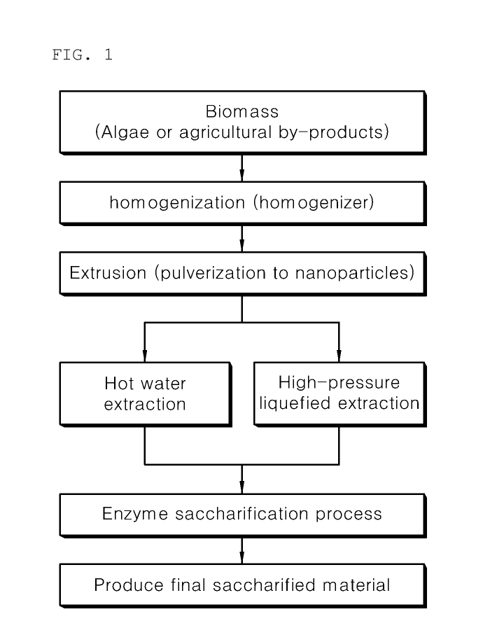

### BEST MODE

### Mode for Invention

The advantages, the features, and schemes of achieving the advantages and/or features of the present invention will be apparently comprehended by those skilled in the art based on the embodiments, which are detailed later in detail, together with accompanying drawings. However, the present invention is not limited to the following embodiments but includes various applications and modifications. The embodiments will make the disclosure of the present invention complete, and allow those skilled in the art to completely comprehend the scope of the present invention. The present invention is only defined within the scope of accompanying claims.

Hereinafter, a method of saccharifying algae or agricultural by-products according to the present invention will be described in detail.

According to the present invention, the algae includes a mixture including at least one or two selected from the group consisting of red algae, brown algae, green algae and microalgae. The green algae may include ulva pertusa kjellman, seaweed, spirogyra, green laver, sea staghorn, codium minus silva, caulerpa okamurai, or nostocaceae. Preferably, the green algae may include ulva pertusa kjellman. Meanwhile, the red algae may include agar, gelidium elegans, cotonni, pachymeniopsis lanceolata, laver, stone laver, pterocladiella capillacea, acanthopeltis japonica, gloiopeltis tenax, sea string, curely moss, grateloupia elliptica, hypnea charoides, ceramium kondoi, ceramium boydenii, gigartina tenella, seokmok, or grateloupia filicina. The brown algae may include seaweed, laminaria, anlipus japonicus, chordaria flagelliformis, ishige okamurae, whip tube, endarachne binghamiae, ecklonia cava, gom pi, rheum rhabarbarum, costaria costata, sargassum, sargassum horneri, sargassum thunbergill, or hijikia fusiforme.

Meanwhile, the agricultural by-products includes a mixture including at least one or two selected from the group consisting of a barley stem, a rape stem, a sorghum stem, a corn stem, and a rice straw. Preferably, the agricultural by-products may include the barley stem or the rape stem.

First, the algae or agricultural by-products are homogenized and crushed. In this case, the homogenized algae or the by-products is put into a homogenizer and rotated. The algae or agricultural by-products are put into distilled water at a concentration of 1%(w/v) to 30%(w/v) to obtain a mixture and rotated by using the homogenizer. In this case, the algae or agricultural by-products are dried and pulverized to the size of 0.1 mm to 10 mm.

The homogenizing is performed by rotating the homogenizer at a rotational speed of 10,000 rpm to 50,000 rpm, preferably, 20,000 rpm to 30,000 rpm. The homogenizing is performed for about 5 minutes to 60 minutes, preferably, 10 minutes to 30 minutes. The algae or agricultural by-products are crushed through the homogenizing process.

Then, the crushed algae or agricultural by-products are extruded at high pressure. The extrusion is performed by pressing pressure of 10,000 psi to 50,000 psi, preferably 20,000 psi to 40,000 psi. The algae or agricultural by-products pass through a pipe having a micro-diameter under high-pressure. The particle size of the algae or agricultural by-products can be reduced to a nano-size by shear stress when the algae or agricultural by-products pass through the pipe. The diameter of the pipe is preferably in the range of 1 μm to 1,000 μm, particularly 10 μm to 500 μm, and more particularly 50 μm to 100 μm.

The extruded algae or agricultural by-products may be additionally subject to the hot water extraction or the high-pressure liquefied extraction at pressure of 100 Mpa to 2,000 MPa. The hot water extraction may be performed using distilled water as extraction solvent in an extraction flask having a cooler. The high-pressure liquefied extraction may be performed by a high-pressure liquefied extraction device that is generally known to those skilled in the art.

The algae or agricultural by-products subject to the pretreatment by the above processes are saccharified by enzyme. The enzyme may include at least one selected from the group consisting of cellulase, amyloglucosidase, β-agalase, β-galactosidase, β-glucosidase, endo-1,4-β-glucanase, α-amylase, and β-amylase. Preferably, the enzyme includes cellulase, amyloglucosidase. Preferably, the enzyme treatment is performed for about 15 hours to 30 hours. If the enzyme treatment time exceeds 30 hours, the yield rate is not increased from enzyme reaction.

The saccharified material can be obtained through the enzyme treatment, and the saccharified material is fermented to prepare bioethanol. In addition, since the saccharified material is not subject to a pretreatment process using chemical ingredients other than water, the saccharified material can be used for a food industrial material. The saccharified material includes monosaccharide, such as glucose, galactose, 3,6-dihydrogalactose, fucose, ramnose, xylose, or mannose, but the present invention is not limited thereto.

Hereinafter, embodiments and experimental examples of the present invention will be described in detail. The embodiments and experimental examples are provided only for illustrative purposes, and the present invention is not limited thereto.

### Embodiment 1

### Saccharification Pretreatment Process of Ulva pertusa kjellman

In order to remove moisture from the ulva pertusa kjellman, after cleaning ulva pertusa kjellman collected from Jeju-do, the ulva pertusa kjellman was dried for 3 days at a temperature of 100° C. in a hot air drier, sealed and stored.

The dried ulva pertusa kjellman was pulverized in size of about 1 mm to 2 mm, put into a distilled water to the extent that the concentration of the ulva pertusa kjellman is 10% (w/v), and mixed with the distilled water. Then, the ulva pertusa kjellman was put into a homogenizer, homogenized at 25,000 rpm for 20 minutes, and crushed. After filtering out upper portions of crushed ulva pertusa kjellman samples by 95% of volume, the sample was extruded by passing the sample through a pipe having a diameter of 100 μm at the pressure of 25,000 psi. The extruded ulva pertusa kjellman was used as a sample of following experimental example 1.

### Embodiment 2

### Saccharification Pretreatment Process of Gulfweed

The saccharification pretreatment process performed in Embodiment 2 was performed similarly to that in embodiment 1 except that gulfweed collected from Jeju-do was dried for use instead of the dried ulva pertusa kjellman.

### Embodiment 3

### Saccharification Pretreatment Process of Barlay Stem

The saccharification pretreatment process performed in Embodiment 3 was performed similarly to that in embodiment 1 except that a barlay stem remaining after harvest was cut to a length of 1 cm, and dried at a normal temperature for one week for use instead of the dried ulva pertusa kjellman.

### Embodiment 4

### Saccharification Pretreatment Process of Rape Stem

The saccharification pretreatment process performed in Embodiment 4 was performed similarly to that in embodiment 1 except that a rape stem was cut to a length of 1 cm, and dried at a normal temperature for one week for use instead of the dried ulva pertusa kjellman.

### Experimental Example 1

### Comparison Between Amounts of Produced Glucoses in Extracts

1) General Hot Water Extraction

A sample was put into an extraction flask having a vertical reflux condenser attached thereto, and extracted at the temperature of 60° C. for 24 hours by using distilled water having the weight, which is 10 times greater than the weight of the sample, as an extraction solvent.

2) High-Pressure Liquefied Extraction

A sample was put into a high-pressure liquefied extraction device, and distilled water having the weight, which is 10 times greater than the weight of the sample, was added therein. Then, an extraction process was performed at the pressure of 1,000 MPa for 30 minutes.

In order to measure amounts of produced glucose (amounts of reduced sugars) of saccharification liquids obtained through the extraction processes, the following experiment was performed.

Each extracted saccharification liquid was put into a 25 ml polyethylene bottle together with 25 mg of cellulose, and 8 ml of 0.15M CH₃COONa (pH 5.0) buffer solution was applied to the mixture. Next, the bottle was closed with a stopper, and put into a shaking water bath. Thereafter, a temperature was maintained at 50° C., and the bottle was slowly shaken for 72 hours while making a reaction. Then, 6 ml of distilled water was applied 1 minutes before the end of the reaction, so that the whole volume of a reaction solution became 14 ml. A predetermined amount of the reaction solution was taken and centrifugated. Then, reduced sugar was quantified through a DNS scheme. After 1 ml of a DNS solution was applied to 100 μl of samples having different concentrations, the mixture was heated at the temperature of 100° C. for 8 minutes. Then, after the mixture was cooled for four minutes, an optical density was measured at 557 nm to measure an amount of produced glucose. When the measured amount of produced glucose is compared with the content of an initial sample, the conversion yield rate of glucose is calculated, and the calculation results are shown in table 1.

TABLE 1

Amount of produced glucose
(%, w/w)

| | Hot Water | High-Pressure Liquefied |
|---|---|---|
| Samples | Extraction | Extraction |
| Embodiment 1 | 5.23 | 8.59 |
| Embodiment 2 | 3.52 | 6.74 |
| Embodiment 3 | 4.47 | 7.88 |
| Embodiment 4 | 5.12 | 9.56 |
| ulva pertusa | 3.03 | 4.50 |
| kjellman | | |
| Gulfweed | 2.31 | 4.42 |
| Barlay Stem | 2.13 | 5.12 |
| Rape Stem | 2.61 | 6.23 |

As shown in Table 1, when comparing with each of an ulva pertusa kjellman, a gulfweed, a barlay stem, and a rape stem subject to the hot water extraction or the high-pressure liquefied extraction without an additional process, an amount of produced glucose is greatly increased in the samples extracted after being subject to the processes of Embodiments 1 to 4.

### Experimental Example 2

### Production of Glucose According to Enzymatic Saccharification Process

After separating a solid matter and a saccharification liquid from an extraction liquid extracted through hot water extraction or high-pressure liquefied extraction in Experimental example 1, an enzymatic saccharification process was performed using the solid material.

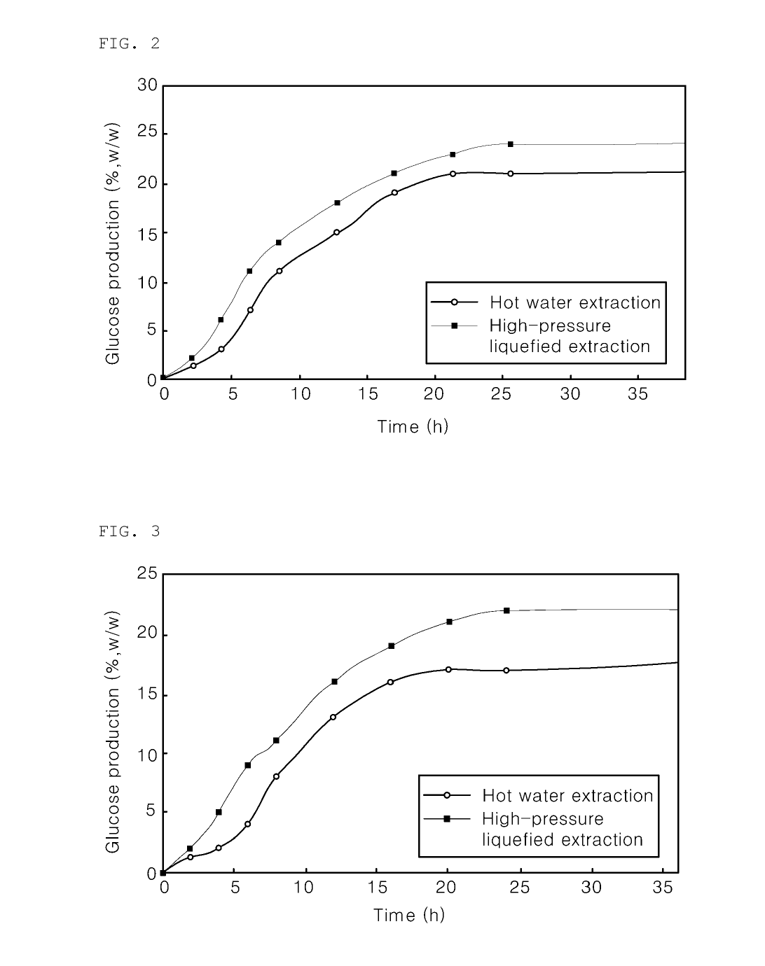

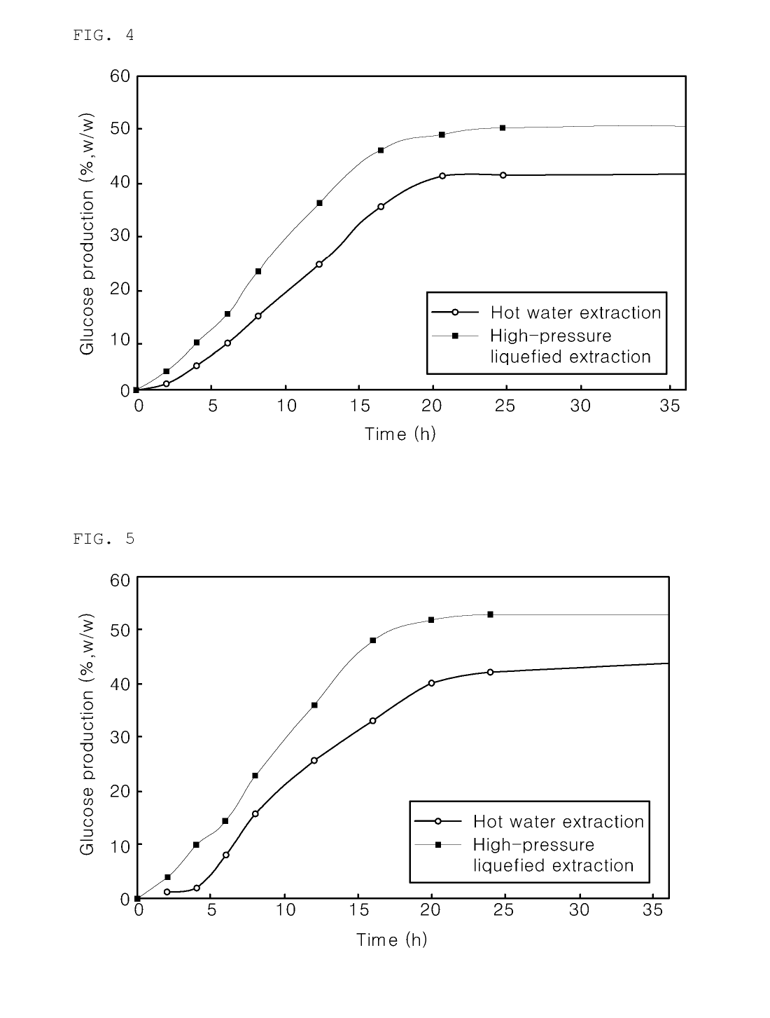

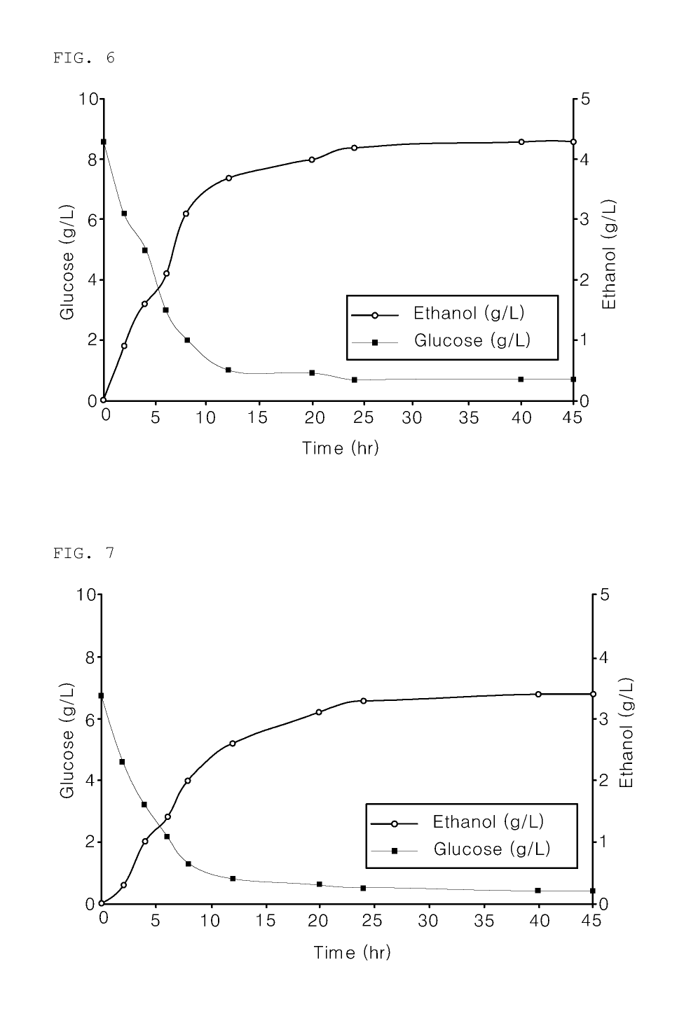

First, after the solid matter was completely dried for at the temperature of 40° C. for 24 hours, the mass of the solid matter was measured to calculate a yield rate. Then, 50 ml of sodium acetate buffer having ph 4.8 and 15 FPU/glucan of cellulose (Celluclast 1.5L, Novozyme 188) were added into a flask. In order to determine an activity degree of enzyme according to times, the enzyme was sampled by 1 ml every predetermined time, and the conversion yield rate according to the time was measured. The measurement result was shown in FIGS. 2 to 5.

As shown in FIGS. 2 to 5, ulva pertusa kjellman and gulfweed represent at least 20% of glucose conversion efficiency, and barley and rape stems represent at least 50% of glucose conversion efficiency. The high-pressure liquefied extraction represents the glucose conversion efficiency higher than that of typical hot water extraction. This is because, as an area of the surface of a fiber is increased through the pre-treatment according to the present invention, the contact area between the enzyme and the fiber is increased, so that a large amount of enzymes can participate in the reaction.

Meanwhile, after about 25 hours have been elapsed, glycosylation efficiency is not increased any more.

### Experimental Example 3

### Comparison Between Amounts of Produced Glucoses by Glucose Fermentation

Sacchromyces cerevisiae (ATCC 24858) serving as fermenting micro-organisms was cultured in a shaking incubator (30° C., 150 rpm) for 24 hours using an YPD (yeast extract 1%, peptone 2%, glucose 2%) culture medium. In this case, water was put into the shaking incubator to culture the sacchromyces cerevisiae in volume of 800 ml. The culture fluid obtained through the culturing process was used in the fermentation.

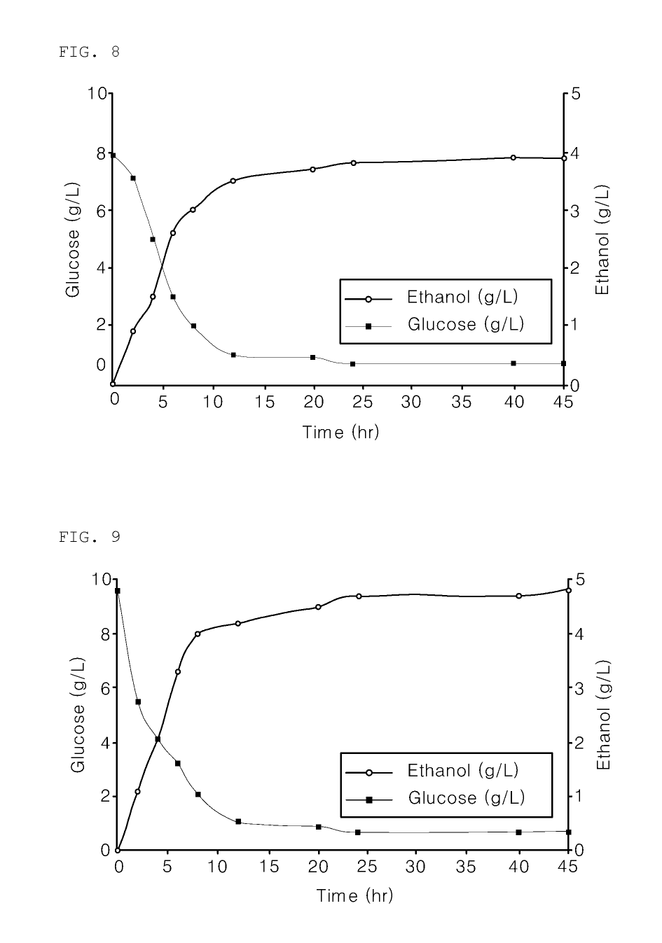

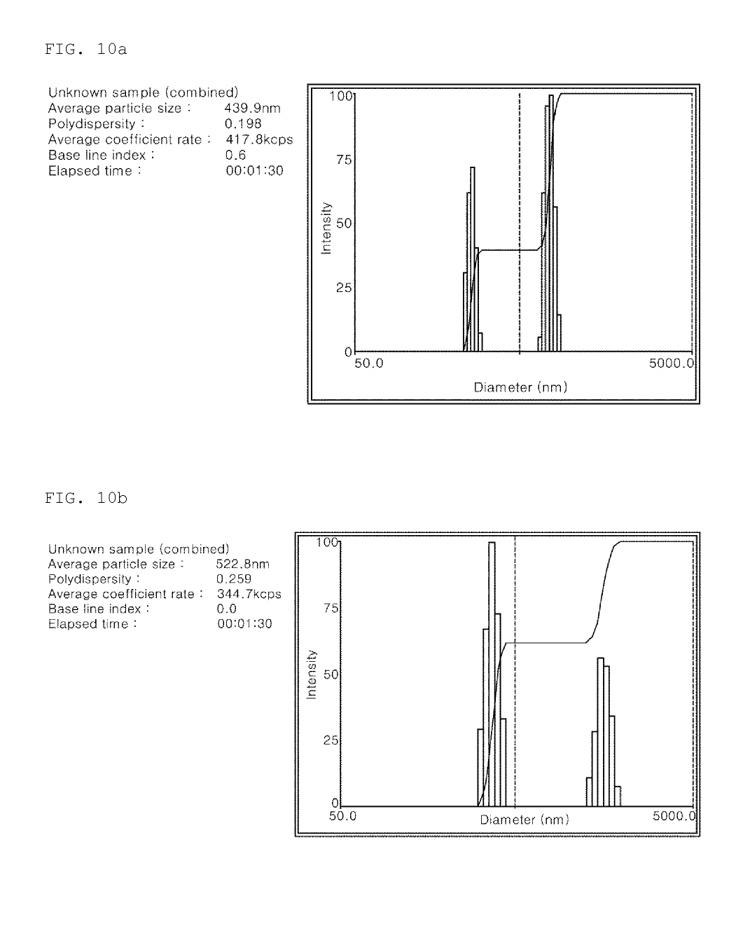

The culture fluid was mixed with the saccharification liquid acquired through the high pressure liquefied extraction in Experiment example 1 and the mixture was fermented at a normal temperature. An amount of ethanol produced according to times is shown in FIGS. 6 to 9.

As shown in FIGS. 6 to 9, the yield rate of the ethanol theoretically reaching the maximum value can be ensured through the fermentation. In addition, since the toxic property against a fermentation strain can be minimized through an eco-friendly process using only pure water, the high yield rate of the ethanol can be obtained.

### Experimental Example 4

### Observation of Particles of Extruded Biomass

In order to observe the size and the surface of particles of a bio-mass extruded according to the embodiments, a dynamic light scattering (DLS) scheme and a scanning electron microscope (SEM) scheme are used.

1) Observation by DLS

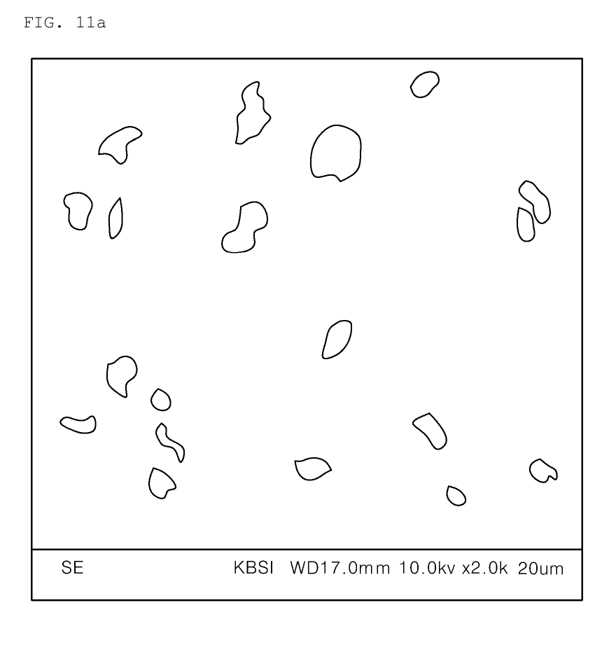

Ulva pertusa kjellman and a rape stem extruded in Embodiments 1 and 4, respectively, were put into cuvettes by 3 ml, respectively and the sizes of the ulva pertusa kjellman and the rape stem were measured at a time interval of 30 seconds for 1 minute 30 seconds by using a DLS nano-particle analyzer, and the analysis result is shown in FIGS. 10a and 10b.

As shown in FIG. 10a, the ulva pertusa kjellman of Embodiment 1 has an average particle size of 439.9 nm. As shown in FIG. 10b, the rape stem of Embodiment 4 has an average particle size of 5222.8 nm. It is recognized from the process of the embodiment that the bio-mass has a nano-size particle.

2) Observation of SEM

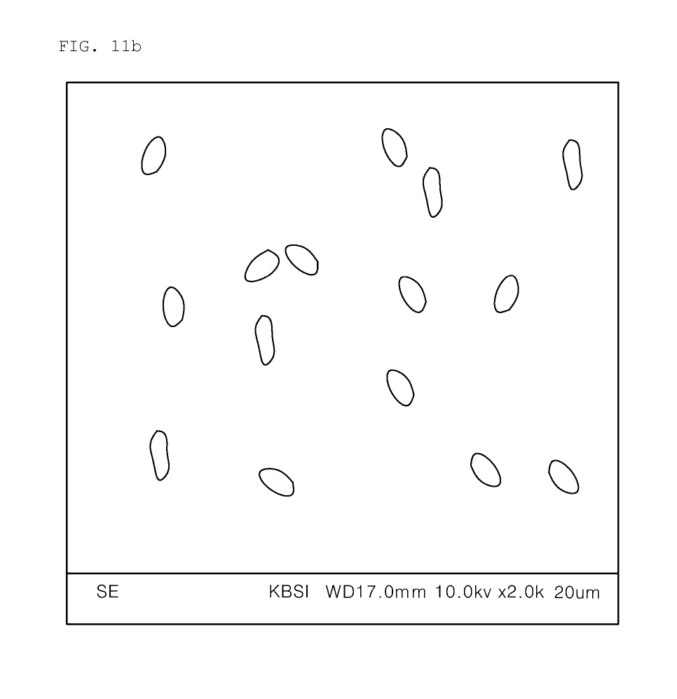

In order to observe a morphology change of a biomass tissue subject to the extrusion process according to the present invention, the surface of the ulva pertusa kjellman in the first embodiment was observed by using a vacuum scanning electron microscope (SEM), and the SEM photography is shown in FIG. 11.

FIG. 11a is an SEM photograph showing a tissue surface of an ulva pertusa kjellman sample crushed using the homogenizer in the first embodiment. FIG. 11b is an SEM photograph showing a tissue surface of an ulva pertusa kjellman sample crushed using a homogenizer in the first embodiment and subject to an extrusion process.

As shown in FIGS. 11a and 11b, the tissue surface of the ulva pertusa kjellman subject to the extrusion process is more destructed to make a great difference from an ulva pertusa kjellman sample that is not subject to the extrusion process, thereby making a difference in extracting glucose.

As described above, although various examples have been illustrated and described, the present disclosure is not limited to the above-mentioned examples and various modifications can be made by those skilled in the art without departing from the scope of the appended claims. In addition, these modified examples should not be appreciated separately from technical spirits or prospects. Therefore, it should be understood that the present invention is not limited to the embodiments described above. The scope of the present invention will be limited by the appended claims. In addition, it will also be apparent to those skilled in the art that variations or modifications from the appended claims and the equivalent concept of the claims are included in the scope of the present invention.

### INDUSTRIAL APPLICABILITY

As described above, according to the present invention, non-biodegradable polymers, such as cellulose, which is a polysaccharide included in biomass, such as algae or agricultural by-products, hemicelluloses, starch, and complex polysaccharide can be hydrolyzed at high glycosylation efficiency through an eco-friendly pretreatment process using water.

In particular, according to the present invention, since only water is used, the neutralization process can be omitted. Further, since the features of the continuous pretreatment process can be represented, processes can be simplified, so that the low-cost and high efficiency can be expected.

In addition, since the saccharified materials produced according to the method of the present invention do not contain materials, such as furuals and furans, to degrade fermentation, the saccharified materials produced according to the method of the present invention can be widely applied to a food industry as well as a bio energy industry.

1. A method of saccharifying algae or agricultural by-products, the method comprising:
1) homogenizing and crushing the algae or agricultural by-products; and
2) extruding the algae or agricultural by-products that are crushed.

2. The method of claim 1, wherein the extruding of the algae or agricultural by-products are performed at pressure in a range of 10,000 psi to 50,000 psi.

3. The method of claim 1, wherein the extruding of the algae or agricultural by-products are performed by applying pressure such that the crushed algae or agricultural by-products pass through a pipe having a diameter in a range of 10 μm to 1,000 μm.

4. The method of claim 1, further comprising rotating a homogenizer to homogenize the algae or agricultural by-products at a rotational rate in a range of 10,000 rpm to 50,000 rpm.

5. The method of claim 1, wherein the homogenizing of the algae or agricultural by-products comprises:
putting the algae or agricultural by-products into distilled water at a concentration of 1%(w/v) to 30%(w/v) to obtain a mixture; and
rotating the mixture using a homogenizer.

6. The method of claim 5, wherein dried algae or agricultural by-products are pulverized to a size in a range of 0.1 mm to 10 mm and mixed with the distilled water.

7. The method of claim 1, further comprising performing hot water extraction for the extruded algae or agricultural by-products or performing high-pressure liquefied extraction for the extruded algae or agricultural by-products at pressure in a range of 100 Mpa to 1,000 Mpa.

8. The method of claim 1, further comprising performing enzyme-treatment for the extruded algae or agricultural by-products.

9. The method of claim 8, wherein the enzyme includes at least one selected from the group consisting of cellulase, amyloglucosidase, β-agalase, β-galactosidase, β-glucosidase, endo-1,4-β-glucanase, α-amylase, and β-amylase.

10. The method of claim 1, wherein the algae includes a mixture including at least one or two selected from the group consisting of red algae, brown algae, green algae and microalgae, and the agricultural by-products includes a mixture including at least one or two selected from the group consisting of a barley stem, a rape stem, a sorghum stem, a corn stem, and a rice straw.

11. A method of preparing bioethanol, the method comprising fermenting a saccharified material obtained through a method of saccharifying algae or agricultural by-products according to one of claims 1 to 10.
